(19) United States
(12) Patent Application Publication
(10) Pub. No.: US 2019/0054098 A1
(43) Pub. Date: Feb. 21, 2019

(54) AGENTS AND METHODS USING THEREOF FOR THE PREVENTION AND TREATMENT OF STEM CELLS SENESCENCE

(71) Applicant: ECOLE POLYTECHNIQUE FEDERALE DE LAUSANNE (EPFL), LAUSANNE (CH)

(72) Inventors: HONGBO ZHANG, GUANGZHOU (CN); KEIR MENZIES, LUSKVILLE QC (CA); JOHAN AUWERX, BUCHILLON (CH); DONGRYEOL RYU, BUSAN (KR)

(21) Appl. No.: 15/758,369

(22) PCT Filed: Sep. 7, 2016
(86) PCT No.: PCT/EP2016/071039

§ 371 Date: Mar. 8, 2018

(30) Foreign Application Priority Data
Sep. 8, 2015 (EP) 15184337.2

(51) Int. Cl. A61K 31/65; A61K 9/00; A61K 31/165; A61L 27/38; A23L 33/10; A61P 43/00

(52) U.S. Cl. CPC A61K 31/65; A61K 9/0053; A61K 31/165; A61L 27/3826; A61K 35/34; A61P 43/00; A61K 9/0029; A61L 27/383; A23L 33/10

(57) ABSTRACT

The present invention relates to agents that prevent or reverse process of stem cell senescence. Further, the invention relates to methods and compositions useful in the prevention and/or treatment of stem senescence.

### FIELD OF THE INVENTION

The present invention relates generally to the field of stem cell medicine and in particular regenerative therapies. In particular, the invention relates to methods and compositions useful in prevention and/or treatment of stem cell senescence.

### BACKGROUND OF THE INVENTION

In adults, tissue homeostasis is highly dependent on adult stem cells (SCs) function in multiple tissues. These adult SCs are not only essential in continuously-proliferating to tissues, such as hematopoietic-, intestinal- and skin-systems, but also in normally quiescent tissues, such as skeletal muscle and brain that require regeneration following damage or with disease (Wagers and Weissman, 2004, Cell, 116: 639). Adult stem cells (SCs) are essential for tissue maintenance and regeneration yet are susceptible to SC senescence during aging that is a decline in adult SC quantity and function. SC senescence is at least partly responsible for the loss of tissue homeostasis and regenerative capacity (Kuilman et al., 2010, Genes & Development, 24: 2463; Lopez-Otin et al., 2013, Cell, 153: 1194).

One example of a tissue where homeostasis depends on adult stem cells is muscle. In skeletal muscle, homeostasis and regeneration depends on the normally quiescent skeletal muscle stem cells (MuSCs), which are activated upon muscle damage to expand and give rise to differentiated progeny that regenerate damaged muscle fibers (Yin et al., 2013, Physiological Reviews, 93: 23; Tabebordbar et al., 2013, Annual Review of Pathology, 8: 441). These responses are blunted in aged muscle due to a quantitative and qualitative decline in MuSCs (Jang et al., 2011, Cold spring Harbor symposia on quantitative biology 76: 1001; Price et al., 2014, Nature Med., 20: 1094). In aging, MuSC dysfunction may be attributed to both extrinsic signals (Conboy et al., 2005, Nature, 433: 760; Chakkalakal et al., 2012, Nature, 490: 335) and/or intrinsic cellular senescence signalling pathways (Sousa-Victor et al., 2014, Nature 506: 316). One general regulator of cellular senescence, cyclin-dependent kinase inhibitor 2A (CDKN2A, p16^{INK4A}), is increasingly expressed in geriatric MuSCs (Burd et al., 2013, Cell, 152: 316), eliciting permanent cell cycle withdrawal and senescence of MuSCs in very old mice (Sousa-Victor et al., supra). However, before this stage, reductions in MuSC number and function can already be observed (Jang et al., supra; Sousa-Victor et al., supra) indicating that MuSC senescence may be initiated at an earlier time point. Several recent reports support the idea that pre-geriatric mice, approximately two-years-old, can exhibit features of MuSC senescence (Price et al., supra; Bernet et al., 2014, Nature Med. 20: 265; Cosgrove et al., 2014, Nature Med. 20: 255; Tierney et al., 2014, Nature Med. 20: 1182; Liu et al., 2013, Cell Rep. 4: 189). However, the early mechanisms that instigate MuSC senescence are still largely unknown.

Another example of tissue where homeostasis depends on adult stem cells is brain tissue. Throughout the lives of all mammals, two distinct populations of neural stem cells (NSCs) are maintained in the brain: one in the subgranular zone (SGZ) of the dentate gyrus (DG) and the other in the subventricular zone (SVZ) bordering the lateral ventricles. NSCs have the ability to self-renew and to differentiate into transient amplifying progenitors (collectively referred to as neural stem/progenitor cells or NSPCs), which in turn undergo cell divisions to produce neurons, oligodendrocytes, or astrocytes (Breunig et al., 2011, Neuron, 4:614-35; Stein et al., 2014, EMBO, J. 33: 1321). In mice, the number of proliferating NSPCs in the SGZ declines exponentially in the first 9 months of life and they are almost completely absent by 24 months of age.

Aging is also associated with impaired oligodendrocyte differentiation and remyelination in response to insult (Stein et al., supra).

Stem cell senescence is involved in a large number of diseases or disorders such as muscle dystrophies (Meregalli, et al., 2012, Curr Gene Ther, 12(3):192-205), myopathies (Cossu, et al., 2000, J Clin Invest, 105 (12):1669-74) neurodegenerative diseases (Upadhyay et al., 2015, Mol Neurobiol, 52(1):610-25), cardiac and skin disorders (Sussman, et al., 2004, Annu Rev Physiol, 66:29-48; Zouboulis et al., 2008, Exp Gerontol, 43(11):986-97).

There is currently no cure or validated therapy for either stem cell senescence nor stem cell senescence disorders induced/linked by other diseases. The available medications and therapies can only slow the course of the disease. For instance, in order to prevent some muscle stem senescence and related muscular dystrophy, some dietary intervention such as creatine, or protein-rich diets, exercise are recommended and the use mesenchymal stem cells transplantation or MuSC transplantation are investigated. For preventing neural stem cell senescence, there is no approved therapy.

Some theoretical potential treatments for stem cell senescence in general have been proposed, but they have not tested in humans (Oh et al., 2014, Nat Med, 20:870-80), such as targeting oxidative stress related reactive oxygen species (ROS) and activating of DNA damage repair pathways.

Therefore, there is a broad need for the development of strategies useful for preventing and/or treating stem cell senescence.

### SUMMARY OF THE INVENTION

The present invention is directed to the unexpected findings that agents such as thiamphenicol (TAP) through the induction of the mitochondrial unfolded protein response (UPR^{mt}) and of prohibitin proteins can directly impact modulating stem cell senescence, in particular the regulation of skeletal muscle stem cell senescence. Therefore, those agents could therefore be utilized to reprogram dysfunctional SCs in aging and disease to improve healthspan in mammals. The invention in particular relates to the unexpected findings that agents such as thiamphenicol (TAP) which also induce cell cycle gene expression in senescent C2C12 myoblasts in vitro.

One aspect of the invention provides an agent selected from thiamphenicol (TAP) or an analogue thereof such as other amphenicols such as chloramphenicol, azidamfenicol and florfenicol and a tetracycline or an analogue thereof, for use in the prevention and/or treatment of diseases or disorders associated with SC senescence and/or for promoting tissue growth and/or repair.

Another aspect of the invention provides a use of an agent selected from thiamphenicol (TAP) or an analogue thereof and tetracycline or an analogue thereof for the preparation of a composition for prevention and/or treatment of diseases or disorders associated with SC senescence and/or for promoting tissue growth and/or repair.

Next aspect of the invention provides a composition comprising an agent selected from thiamphenicol (TAP) or an analogue thereof and tetracycline or an analogue thereof or a mixture thereof and an agent useful in the prevention and/or treatment of diseases, disorders or toxic effects leading to or associated with SC senescence and/or for promoting tissue growth and/or repair.

Another aspect of the invention provides a composition for ex-vivo preservation of cells, tissue or organs comprising at least one agent selected from an agent selected from thiamphenicol (TAP) or an analogue thereof and tetracycline or an analogue thereof or a mixture thereof.

Another aspect of the invention provides a food supplement comprising thiamphenicol (TAP) or an analogue thereof or a mixture thereof.

Another aspect of the invention provides a stem cell culture medium or a composition for preservation of cells, organs and tissues comprising an agent selected from thiamphenicol (TAP) or an analogue thereof and tetracycline or an analogue thereof or a mixture thereof.

Another aspect of the invention provides a method of preventing and/or treating of diseases or disorders associated with SC senescence in a subject and/or promoting tissue growth and/or repair, said method comprising administering an effective amount of an agent selected from thiamphenicol (TAP) or an analogue thereof and tetracycline or an analogue thereof, a mixture thereof or a pharmaceutical composition thereof in a subject.

Another aspect of the invention provides a method of maintaining and/or extending stemness of a stem cell population comprising contacting a stem cell population or a stem cell containing sample with a composition of the invention.

Another aspect of the invention provides a method for promoting tissue growth and/or repair, in particular for improving cell/tissue survival, comprising using a composition or a method of the invention.

Another aspect of the invention provides an ex-vivo method for preparing a graft sample in view of promoting tissue growth and/or repair, in particular for improving cell/tissue survival and engraftment ability after grafting said graft sample.

A further aspect of the invention provides a kit for stem cell culture or for ex-vivo preservation of cells, organs and tissues comprising at least one agent selected from thiamphenicol (TAP) or an analogue thereof and tetracycline or an analogue thereof or a mixture thereof or a composition of the invention with instructions of use.

Another aspect of the invention provides a method of cell-based therapy, said method comprising administering, grafting a stem cell composition of the invention. Said stem cells could be prepared according to a method of the invention.

### DESCRIPTION OF THE FIGURES

FIG. 1 shows reduction of mitochondrial content and oxidative respiration in MuSCs during aging. A: GSEA demonstrates up- and downregulated signaling pathways in MuSCs from two-year-old mice, compared to four-month-old mice. Signaling pathways are ranked on the basis of normalized enrichment scores (NESs); positive and negative NESs indicate down- or upregulation in aged MuSCs, respectively. Specific pathways related to MuSC function are marked in black. B: Top 10 ranked downregulated pathways in MuSCs from aged animals (GSE47177), based on gene ontology (GO) enrichment. Pathways are ranked by familywise error rate (FWER) p values. The top 5 significant down-regulated pathways are marked in grey. C: Area-proportional Venn diagram representing 113 common genes between the significantly downregulated genes (p<0.05) in MuSC transcriptomes originating from aged mice (GSE47177 and GSE47401), and genes from the human mitochondrial transcriptome. D: Pie chart illustrating the percent composition of the common 113 mitochondrial genes found in C. TXN, transcription, TLN, translation. E, Custom gene-set analysis showing enrichment of OXPHOS, TCA cycle and UPR^{mt} related transcripts from MuSCs of young (Y) and aged (A) mice obtained from three independent data sets (GSE47177, GSE47401 and GSE47104). Roman numerals indicate corresponding OXPHOS complexes.

FIG. 2 shows reduction of mitochondrial content and oxidative respiration in MuSCs during aging. A-D: MuSCs were isolated from young (3 months old) and aged (22-24 months old) C57BL/6J mice either freshly (A, C and D) or under in vitro cell culture for three generations (B) A: qPCR validation of transcriptional changes in mitochondrial genes of freshly sorted MuSCs. B: OCR in isolated primary MuSCs, cultured in vitro for three generations. C-D: Relative gene expression for UPR^{mt} genes (C) and cell senescence markers (D) in freshly sorted MuSCs. Data are normalized to 36b4 mRNA transcript levels. All data are shown as mean±s.e.m. A-D, n=6 mice per group. *P<0.05, **P<0.01. All statistical significance was calculated by Student's t test.

FIG. 3 shows expression of prohibitins and cell cycle related genes in C2C12 myoblasts following different treatment periods with 50 μg/ml TAP, which induces a mitonuclear imbalance and UPR^{mt} and cell cycle regulators.

### DETAILED DESCRIPTION

As used herein “adult stem cells” or “SCs” or “somatic stem cells” refers to undifferentiated cells, found throughout the body after development, capable of self-renewal (ability of multiply by cell division while still maintaining cell's undifferentiated state). The function of said cells is to replenish dying cells and regenerate damaged tissues of the organ from which they originate, potentially regenerating the entire organ from a few cells. This term encompasses stem cells of muscle, neural, cardiac, pulmonary, hepatic, renal, muscle, skin, intestinal, colonic, pancreatic or bone origin.

Specific example of adult stem cells is, as used herein “skeletal muscle stem cells” or “MuSCs” or “satellite cells” that refers to adult stem cells of muscle, having the capacity to self-renew and to differentiate into myocytes, which fuse among each other or with the existing myofibers to compose the muscle fiber units. The known markers of MuSCs include, but are not limited to several transcription factors PAX7, MYF5, PAX3 and cell surface antigens CD34, Integrin α7, Sca-1 and M-Cadherin.

As used herein “cardiac stem cell” or “CSCs” that refers to endogenous adult heart stem cells. They are tissue-specific stem progenitor cells harbored within the adult mammalian heart. CSCs are clonogenic, self-renewing and multipotent in vitro and in vivo, capable of generating the three major cell types of the myocardium: myocytes, smooth muscle and endothelial vascular cells. Cell membrane markers (Sca-1, Abcg-2, Flk-1) and transcription factors (Isl-1, Nkx2.5, GATA4) are used to their identification (Ellison et al., 2010, Front Biosci, 64: 661-73).

As used herein “neural stem cell” or “NSCs” that refers to adult stem cells in nervous system. In mammals, there are found in the subgranular zone (SGZ) of the dentate gyrus (DG) and the other in the subventricular zone (SVZ) bordering the lateral ventricles. NSCs have the ability to self-renew and to differentiate into neurons, oligodendrocytes, or astrocytes. Cell proliferation markers such as Ki67 and PCNA are always used to distinguish NSCs in brain.

As used herein “skin stem cell” or “SSCs” that refers to tissue specific stem cells in skin. In mammals, at least three types of adult stem cells are residing in the skin: epidermal, hair follicle, and melanocyte stem cell. SSCs ensure the maintenance of adult skin homeostasis, hair color and hair regeneration (Solanas et al., 2013, Nat Rev Mol Cell Biol., 14(11): 737-47). Integrin α6, β1 are the markers of skin epidermal stem cells. CD34, Nestin, Sox9, LGR5 are used as hair folic stem cell (hair germ) markers. c-Kit, TRP2, PAX3 are the well-used markers of melanocyte stem cells.

As used herein “SC senescence” refers to a stable and irreversible loss of proliferative capacity (stable cell cycle arrest), despite continued viability and metabolic activity. Specific markers of SC senescence include, among others, β-galactosidase activation, H2AX phosphorylation, downregulation of cell cycle regulators (Mki67, Cdk4, Ccnd1, Cdkn1a) and induction of inflammatory factors (IL6 and IL18) (Kuilman et al., supra; Lopez-Otin et al., supra). Cdkn1a and Cdkn2a are amongst the general regulators of cellular senescence whose expression is increased in geriatric MuSCs (Burd et al., supra; Lopez-Otin et al., supra).

The expressions “stem cell senescence” or “stem cell senescence related disorders or diseases” include skeletal muscle stem cells senescence, cardiac muscle stem cell senescence, neural stem cells senescence, skin stem cell senescence.

The expression “skeletal muscle stem cell senescence” includes muscle dystrophy diseases, includes Duchenne's muscular dystrophy (DMD), Becker's muscular dystrophy (BMD), Congenital muscular dystrophy, Distal muscular dystrophy, Emery-Dreifuss' muscular dystrophy, Facio-scapulo-humeral muscular dystrophy, Limb-girdle muscular dystrophy, Myotonic muscular dystrophy and Oculopharyngeal muscular dystrophy. It further includes other inherited myopathies, such as myotonia, congenital myopathies (includes nemaline myopathy, multi/minicore myopathy, centronuclear myopathy), mitochondrial myopathies, metabolic myopathies (includes glycogen storage diseases and lipid storage disorder), inflammatory myopathies, such as dermatomyositis, polymyositis, inclusion body myositis and auto-immune myositis, muscle wasting induced by nutritional deficiencies, or other diseases, such as chronic obstructive pulmonary disease (COPD), chronic inflammatory syndromes, and cachexia of cancer. It further includes muscle frailty and sarcopenia in aging (Sousa-Victor et al., supra) and other acquired myopathies, such as drug/toxic agents-induced myopathy, alcoholic myopathy, myositis ossificans, rhabdomyolysis and myoglobinurias.

The expression “cardiac muscle stem cell senescence” includes senescence of the cardiac muscle resulting from a heart attack and myocardial infarction, primary cardiomyopathies, including genetic (e.g. hypertrophic cardiomyopathy, dilated cardiomyopathy, restrictive cardiomyopathy) and acquired forms (e.g. myocarditis) of cardiomyopathy, secondary cardiomyopathies, such as seen within the context of metabolic (e.g. hematochromatosis, obesity) and endocrine (e.g. diabetic and hyperthyroidy) diseases, treatments that are toxic to the heart (e.g. with anthracyclines, such as doxorubicin), coronary artery disease (e.g. post myocardial infarction), neuromuscular disease (e.g. muscular dystrophy), heart failure, stroke, heart valve problems and arrhythmias.

The expression “neural stem cell senescence” includes senescence of the neural stem cells neuro-degenerative diseases such as Alzheimer's disease (AD), Parkinson's disease (PD), Amyotrophic lateral sclerosis (ALS) and Huntington's disease (HD), neural dysfunction accompanying aging, such as neural dysfunction indicated as loss of function of sight, hearing, taste, small, and feeling; loss of voluntary and involuntary functions, loss of function to think and reason. It further includes myasthenia gravis.

The expression “skin stem cell senescence” includes senescence of epidermal, hair follicle and melanocyte stem cells such as involved in skin homeostasis and hair regeneration dysfunction, impaired skin regeneration after injury or trauma, as well as skin transplantation (Blanpain et al., 2006, Annu Rev Cell Dev Biol., 22:339-73). It further includes dermatoporosis in aging, including, but not limited to hair loss and greying, reduction of skin thickness, strength, moist, and elasticity and the increase of skin injury. It further includes vitiligo.

According to a particular aspect, stem cells and stem cell containing samples for graft purposes are allogeneic and autologous.

The term “cell-based therapy” or “cell-based tissue regeneration” include cell replacement therapies making use of allogenic or autologous stem cells, or in the direct induction of tissue regeneration by in situ stimulation of resident stem cells (e.g. inducing resident stem cells mobilization and differentiation for repair), as alternatives to surgical interventions and organ/tissue transplantation. Methods and compositions according to invention can be advantageously used in methods of “cell-based therapy” or “cell-based tissue regeneration” methods used to produce differentiated tissue from progenitor cells or stem cells such as stem cells of muscle, neural, cardiac, pulmonary, hepatic, renal, skin, intestinal, colonic, pancreatic or bone origin as listed herein.

As defined herewith, “mitochondrial unfolded protein response” or “UPR^{mt}” refers to a cellular stress response related to the mitochondria. The UPR^{mt} is induced by mitochondrial stress signals subsequently activating a nuclear transcriptional response. For example, in C. elegans, expression of mitochondrial chaperones HSP-6 (mitochondrial HSP-70 in mammals) is increased, aimed to restore protein homeostasis (also known as proteostasis) in mitochondria. In mammals, modified expression of mitochondrial chaperones Hsp60, Hsp10 as well as proteases such as Clpp are both induced. By either supporting protein fording or misfolded protein degradation, both of them help to achieve the mitochondrial protein homeostasis (also known as proteostasis) (Jovaisaite et al., 2015, Curr Opin Cell Biol., 33: 74-81).

As used herein, the term “stem cell containing sample” comprises any ex-vivo sample isolated from a source of said stem cell (e.g. skeletal muscle, brain and skin sample).

As used herein, the term “stem cell culture medium” refers to any standard cell stem cell culture medium, optionally comprising appropriate differentiation factors, the nature of which may be adapted to the nature of the cell, in particular culture medium suitable for stem cell expansion such as for example culture media described in the following examples or described in Boitano et al., 2010, Science 329, 1345-8.

According to an embodiment, the cell expansion culture medium comprises standard cocktails of cytokines and growth factors. The cocktails of cytokines and growth factors can be used with or without supporting stromal feeder or mesenchymal cells can comprise, but are not restricted to: SCF, TPO, Flt3-L, FGF-1, IGF1, IGFBP2, IL-3, IL-6, G-CSF, M-CSF, GM-CSF, EPO, oncostatin-M, EGF, PDGF-AB, angiopoietin and angiopoietin-like family including Angl5, prostaglandins and eicosanoids including PGE2, Aryl hydrocarbon (AhR) receptor inhibitors such as StemRegenin1 (SR1) and LGC006 (Boitano et al., 2010, supra). For example, neural stem cells can be used in a culture medium further comprising antagonists to TGF beta, dorsomorphin, as differentiation factors. Heart or lung stem cells can be used in a culture medium containing fetal serum (FS), fetal serum protein extracts, myogenic regulatory factors (MRF), stem cell factor (SCF), insulin-like growth factor (IGF), leukemia-inhibitory factor (LIF), angiopoietin (Ang), colony stimulating factors (CSF), various interleukins (IL), and/or other pre-selected differentiation factors. Skeletal muscle stem cells can be used in culture medium containing recombinant human fibroblast growth factor (rhFGF) and a variety of inflammatory factors.

According to a particular aspect, the medium for reservation of cells, organs and tissues according to the invention may further comprise other biological active extractions, such as chicken embryo extract.

As used herein, the term “thiamphenicol analogs” includes amphenicols such as chloramphenicol. Amphenicols are antibiotics with a phenylpropanoid structure such as chloramphenicol, azidamfenicol and florfenicol.

As used therein, the term “tetracyclines” includes doxocycline and minocycline.

As used herein, the term “SCs cell depleted subjects” mean subjects presenting a significant reduced quantity and quality of tissue specific adult stem cells. Specifically, the reduction of MuSCs, neural stem cells and skin stem cells (epidermal, hair follicle and melanocyte stem cells) is observed in aged human subjects, as well as the reduction of MuSCs in muscle dystrophy patients due to the continuous muscle regeneration, which induces the exhausting of MuSCs.

As used herein, “treatment” and “treating” and the like generally mean obtaining a desired pharmacological and physiological effect. The effect may be prophylactic in terms of preventing or partially preventing a disease, symptom or condition thereof and/or may be therapeutic in terms of a partial or complete cure of a disease, condition, symptom or adverse effect attributed to the disease. The term “treatment” as used herein covers any treatment of a disease in a mammal, particularly a human, and includes: (a) preventing the disease from occurring in a subject which may be predisposed to the disease but has not yet been diagnosed as having it for example based on familial history or age; (b) inhibiting the disease, i.e., arresting its development; or relieving the disease, i.e., causing regression of the disease and/or its symptoms or conditions such as improvement or remediation of damage. In particular, a method according to the invention is useful in the maintenance and/or extension of stemness of stem cell population; prevention of cell senescence/apoptosis of stem cell populations; maintenance and/or prevention of the reduction of stem cell proliferation/cell cycle progress; maintenance and or prevention of the reduction of differentiation potential of stem cell population.

The term “subject” as used herein refers to mammals. For example, mammals contemplated by the present invention include human, primates, domesticated animals such as dogs, cats, cattle, sheep, pigs, horses, laboratory rodents and the like.

The term “efficacy” of a treatment or method according to the invention can be measured based on changes in the course of disease or condition in response to a use or a method according to the invention. For example, the efficacy of a treatment or method according to the invention can be measured in MuSCs through the measurement of muscle damage parameters from blood biochemical measurements of creatine kinase, aspartate aminotransferase and total protein levels. The efficacy of a treatment or to method according to the invention can be measured through the measurement of muscle force, as well as immunostaining of MuSCs number and analysis of the regeneration of the damaged muscle. The efficacy of a treatment or method according to the invention can be measured in NSC through measurement of NSC quantity and the number of newly generated neurons in central neural system, as well as the beneficial impact on the development of disease phenotypes in neurodegenerative diseases. The efficacy of a treatment or method according to the invention can be measured in SSC through measurement of epidermal, hair follicle and melanocyte stem cells number, of the hair follicle cycle in aging and of the recovery from UV/burn/trauma-induced skin damage.

The terms “effective amount”, “therapeutic effective amount”, and “prophylactic effective amount” refer to a dosage of a compound or composition effective for eliciting a desired effect, commensurate with a reasonable benefit/risk ratio and will vary from subject to subject, depending, for example, on species, age, and general condition of a subject, severity of the side effects or disorder, identity of the particular compound(s), mode of administration, and the like. In certain embodiments, the desired dosage can be delivered using multiple administrations. Those terms as used herein may also refer to an amount effective at bringing about a desired in vivo effect in an animal, preferably, a human, such as induction of proliferation of tissue specific stem cells and the faster tissue regeneration.

The efficacy of a treatment or method according to the invention can be measured by determining the level of cell maturation or of cell differentiation in the cell culture medium using standard methods in the art, including visual observation by microscopy, detection of markers which are specific for the targeted differentiated tissue by immunological staining or blotting and by molecular assays of mRNA, chromatin, nuclear DNA, mtDNA, or microRNA.

Use According to the Invention

According to an embodiment, the invention provides an agent selected from thiamphenicol (TAP) or an analogue thereof and tetracycline or an analogue thereof or composition thereof for use in the prevention and/or treatment of a disease or disorder associated with SC senescence and/or decreased SCs number and/or treatment of diseases or disorders associated with SC senescence and/or for promoting tissue growth and/or repair.

According to another embodiment, the invention provides a use of an agent selected from thiamphenicol (TAP) or an analogue thereof and tetracycline or an analogue thereof for the preparation of a composition or a food supplement for the prevention and/or treatment of a disease or disorder associated with associated with SC senescence and/or decreased SCs number and/or for promoting tissue growth and/or repair.

According to another embodiment, the invention provides an agent selected from thiamphenicol (TAP) or an analogue thereof and tetracycline or an analogue thereof or composition thereof for use in the treatment of an injured tissue or organ, notably after an injury or trauma.

According to another embodiment, the invention provides an agent selected from thiamphenicol (TAP) or an analogue thereof and tetracycline or an analogue thereof or composition thereof for use in the treatment of skeletal muscle stem cell senescence such as muscle dystrophy, muscle myopathy and muscle frailty.

According to another embodiment, the invention provides an agent selected from thiamphenicol (TAP) or an analogue thereof and tetracycline or an analogue thereof or composition thereof for use in the treatment of a muscle frailty and sarcopenia.

According to another embodiment, the invention provides an agent selected from thiamphenicol (TAP) or an analogue thereof and tetracycline or an analogue thereof or composition thereof for use in the treatment of neural degenerative diseases, such as, for example Alzheimer's disease (AD), Parkinson's disease (PD), Amyotrophic lateral sclerosis (ALS) and Huntington's disease (HD).

According to another embodiment, the invention provides an agent selected from thiamphenicol (TAP) or an analogue thereof and tetracycline or an analogue thereof or composition thereof for use in the treatment of neural disorders due to loss of neuron that accompany with aging, such as for example loss of function of sight, hearing, taste, small, and feeling; loss of voluntary and involuntary functions, loss of function to think and reason.

According to another embodiment, the invention provides an agent selected from thiamphenicol (TAP) or an analogue thereof and tetracycline or an analogue thereof or composition thereof for use in cardiac muscle regeneration, for example in coronary artery disease, after myocardial infarction, and in primary and secondary forms of cardiomyopathy.

According to another embodiment, the invention provides an agent selected from thiamphenicol (TAP) or an analogue thereof and tetracycline or an analogue thereof for use in skin regeneration, for example in the treatment of skin injury, burns or trauma and in skin transplantation.

According to another embodiment, the invention provides an agent selected from thiamphenicol (TAP) or an analogue thereof and tetracycline or an analogue thereof or composition thereof for use in skin stem cell senescence such as in loss of function of the skin in aging.

According to another embodiment, the invention provides a method for promoting tissue growth, in particular for improving cell/tissue survival or increasing engraftment ability of a SC containing sample, said method comprising contacting or administering to a SC containing sample (e.g. a stem cell, an isolated tissue or organ, a graft sample in culture) before transplantation/grafting to a mammal in need thereof (ex-vivo), an agent selected from thiamphenicol (TAP) or an analogue thereof and tetracycline or an analogue thereof or composition thereof in an amount effective to stimulate maintenance, proliferation, survival of cells and/or maintain and/or extend stemness of stem cell population.

A method of preparation of a cell composition for cell-based therapy comprising a step of contacting or administering to a stem cell an agent selected from thiamphenicol (TAP) or an analogue thereof and tetracycline or an analogue thereof or composition thereof.

According to a further embodiment of the invention, is provided a kit for stem cell culture or tissue or organ graft preparation or for organ or tissue preservation comprising an agent selected from thiamphenicol (TAP) or an analogue thereof and tetracycline or an analogue thereof or a mixture thereof or a formulations thereof and instruction of use.

A method of preparation of a graft sample (e.g. organ, cell or tissue) comprising a step of contacting said graft organ, cell or tissue with an agent selected from thiamphenicol (TAP) or an analogue thereof and tetracycline or an analogue thereof or composition thereof.

According to a further embodiment, the invention provides a method of prevention and/or treatment of diseases or disorders associated with SC senescence decreased SCs number, said method comprising grafting of a cell composition or graft sample prepared according to methods described herein.

According to a particular embodiment, the invention provides a method for promoting tissue growth and/or repair in a subject in need thereof, said method comprising administering an effective amount of an agent selected from thiamphenicol (TAP) or an analogue thereof and tetracycline or an analogue thereof or composition thereof in said subject.

According to an embodiment, the invention provides a method of preventing and/or treating SC senescence and/or decreased SCs number in a subject, said method comprising administering an effective amount of an agent selected from thiamphenicol (TAP) or an analogue thereof and tetracycline or an analogue thereof or composition thereof in said subject.

According to a particular aspect, a method of the invention is an ex-vivo method useful for maintaining and/or extending stemness of a stem cell population.

According to another particular embodiment of the invention, is provided a method for ex-vivo preparing a graft sample comprising the steps of:
a) providing a SC-containing sample in a stem cell culture medium;
b) contacting said SC sample with at least an agent selected from thiamphenicol (TAP) or an analogue thereof and tetracycline or an analogue thereof or a mixture thereof in an amount effective to stimulate the survival and the maintenance of the stemness of the stem cells within the sample as compared to a sample in absence of said at least an agent selected from thiamphenicol (TAP) or an analogue thereof and tetracycline or an analogue thereof or a mixture thereof.

According to a further aspect, said SC-containing sample is further combined with a tissue or organ to be grafted, before grafting. Isolated skeletal muscle stem cells can be treated with one agent of the invention alone, such as TAP alone, or in combination with another useful to proliferation and maintenance of stemness.

According to a particular aspect, the method of graft sample preparation of the invention is useful for promoting tissue growth and/or repair following graft sample grafting.

According to another particular embodiment, is provided an ex-vivo method of the invention, wherein stemness (e.g. self-renewing capacity of SCs) is assessed by quantifying stemness markers such as transcription factors PAX7, MYF5, PAX3 and cell surface antigens CD34, Integrin α7, Sca-1 and M-Cadherin of the cell preparation obtained after step b).

According to another embodiment, is provided a stem cell culture medium comprising an agent selected from thiamphenicol (TAP) or an analogue thereof and tetracycline or an analogue thereof or a mixture thereof, optionally further comprising a cocktail of cytokines and growth factors useful for stem cell expansion. In a further particular embodiment, is provided a stem cell culture medium for skeletal muscle stem cell expansion such as skeletal muscle stem cells isolated from aged persons for subsequent transplantation.

Cells and graft samples obtained by a method according to the invention can be formulated for clinical stem cell or graft or tissue transplantation, or for augmentation stem function or for cell-based therapy in a subject in need thereof.

Compositions According to the Invention

The agents of the invention or formulations thereof may be administered as a pharmaceutical formulation or a food supplement or may be formulated as stem cell culture or organ preservation media, which can contain one or more agents according to the invention in any form described herein. The compositions according to the invention, together with a conventionally employed adjuvant, carrier, diluent or excipient may be placed into the form of pharmaceutical compositions and unit dosages thereof, and in such form may be employed as solids, such as tablets or filled capsules, or liquids such as solutions, suspensions, emulsions, elixirs, or capsules filled with the same, all for oral use, or in the form of sterile injectable solutions for parenteral (including subcutaneous) use by injection or continuous infusion. Injectable compositions are typically based upon injectable sterile saline or phosphate-buffered saline or other injectable carriers known in the art. Such pharmaceutical compositions and unit dosage forms thereof may comprise ingredients in conventional proportions, with or without additional active compounds or principles, and such unit dosage forms may contain any suitable effective amount of the active ingredient commensurate with the intended daily dosage range to be employed.

Compositions of this invention may be liquid formulations including, but not limited to, aqueous or oily suspensions, solutions, emulsions, syrups, and elixirs. The compositions may also be formulated as a dry product for reconstitution with water or other suitable vehicle before use. Such liquid preparations may contain additives including, but not limited to, suspending agents, emulsifying agents, non-aqueous vehicles and preservatives. Suspending agents include, but are not limited to, sorbitol syrup, methyl cellulose, glucose/sugar syrup, gelatin, hydroxyethyl cellulose, carboxymethyl cellulose, aluminum stearate gel, and hydrogenated edible fats. Emulsifying agents include, but are not limited to, lecithin, sorbitan monooleate, and acacia. Preservatives include, but are not limited to, methyl or propyl p-hydroxybenzoate and sorbic acid. Dispersing or wetting agents include but are not limited to poly(ethylene glycol), glycerol, bovine serum albumin, Tween®, Span®.

Compositions of this invention may also be formulated as a depot preparation, which may be administered by implantation or by intramuscular injection.

Solid compositions of this invention may be in the form of tablets or lozenges formulated in a conventional manner. For example, tablets and capsules for oral administration may contain conventional excipients including, but not limited to, binding agents, fillers, lubricants, disintegrants and wetting agents. Binding agents include, but are not limited to, syrup, accacia, gelatin, sorbitol, tragacanth, mucilage of starch and polyvinylpyrrolidone. Fillers include, but are not limited to, lactose, sugar, microcrystalline cellulose, maize starch, calcium phosphate, and sorbitol. Lubricants include, but are not limited to, magnesium stearate, stearic acid, talc, polyethylene glycol, and silica. Disintegrants include, but are not limited to, potato starch and sodium starch glycollate. Wetting agents include, but are not limited to, sodium lauryl sulfate. Tablets may be coated according to methods well known in the art.

The compounds of this invention can also be administered in sustained release forms or from sustained release drug delivery system

For post-transplant or post-chemotherapy patients parenteral administration will be preferred, as post-chemotherapy mucitis often impairs all oral intake, some patients even requiring parenteral nutrition. According to a particular embodiment, compositions according to the invention are for intravenous use.

According to a particular aspect, the formulations of the invention are oral formulations.

According to a particular embodiment, compositions according to the invention are food supplement.

In another particular aspect, the compositions according to the invention are adapted for delivery by repeated administration.

In another particular aspect, the compositions according to the invention are adapted for the stem cell culture or graft preparation or transplantation.

According to a particular embodiment, compositions of the invention are veterinary compositions.

According to a particular embodiment, compositions of the invention are adapted for topical and cosmetic delivery.

Further materials as well as formulation processing techniques and the like are set out in Part 5 of Remington's “The Science and Practice of Pharmacy”, 22^{nd} Edition, 2012, University of the Sciences in Philadelphia, Lippincott Williams & Wilkins, which is incorporated herein by reference.

Mode of Administration

Agents of the invention or formulations thereof may be administered in any manner including orally, parenterally, intravenously, rectally, or combinations thereof. Parenteral administration includes, but is not limited to, intravenous, intra-arterial, intra-peritoneal, subcutaneous and intramuscular. The compositions of this invention may also be administered in the form of an implant, which allows slow release of the compositions as well as a slow controlled i.v. infusion.

According to a particular aspect, agents or formulations thereof are to be administered by injection.

According to a particular aspect, agents or formulation thereof are to be administered orally.

According to one aspect, an oral dosage rate of thiamphenicol ranging from about 5 to about 15 mg/kg/day.

The dosage administered, as single or multiple doses, to an individual will vary depending upon a variety of factors, including pharmacokinetic properties, patient conditions and characteristics (sex, age, body weight, health, size), extent of symptoms, concurrent treatments, frequency of treatment and the effect desired.

Combination

According to the invention, the agents or formulations thereof, including pharmaceutical formulations thereof can be administered alone or in combination with a co-agent (e.g. multiple drug regimens) useful for preventing or treating a disease or disorder associated with SC senescence and/or decreased SCs number.

According to the invention, agents of the invention or formulations thereof, including pharmaceutical formulations thereof can be administered alone or in combination with a co-agent (e.g. multiple drug regimens) useful for graft tissue improvement, in particular for promoting tissue growth and/or repair, in particular for improving cell/tissue survival.

According to the invention, agents of the invention or formulations thereof, can be administered to a subject prior to, simultaneously or sequentially with other therapeutic regimens or co-agents useful for preventing or treating a disease or disorder associated with SC senescence and/or decreased SCs number or useful for promoting tissue growth and/or repair.

A compound of the invention or a formulation thereof according to the invention that is administered simultaneously with said co-agents can be administered in the same or different composition(s) and by the same or different route(s) of administration.

According to a particular embodiment, is provided a formulation comprising an agent selected from thiamphenicol (TAP) or an analogue thereof and tetracycline or an analogue thereof or a mixture thereof, combined with at least one co-agent useful for preventing or treating a disease or disorder associated with SC senescence and/or decreased SCs number or useful for promoting tissue growth and/or repair.

Patients

According to an embodiment, subjects according to the invention are subjects suffering from disease or disorders associated with stem cells senescence, in particular cardiovascular diseases, muscle dystrophy diseases (DMD, BMD, etc), muscle frailty, ischemia-reperfusion injury, neurodegenerative disorders, such as Alzheimer's disease (AD), Parkinson's disease (PD), Amyotrophic lateral sclerosis (ALS) and Huntington's disease (HD), traumatic injury and xenograft rejection, neuron deficiency of the nervous system, radiation damage, cancer and skin injury.

According to an embodiment, subjects according to the invention are subjects suffering from disease or disorders associated with skeletal muscle stem cells senescence.

According to a further embodiment, subjects according to the invention are subjects suffering from disease or disorders associated with skeletal muscle stem cells senescence.

According to an embodiment, subjects according to the invention are subjects suffering from disease or disorders associated with neural stem cell senescence.

According to an embodiment, subjects according to the invention are subjects suffering from disease or disorders associated with cardiac stem cell senescence.

According to an embodiment, subjects according to the invention are subjects suffering from disease or disorders associated with skin stem cell senescence.

In a particular embodiment, subjects according to the invention are subjects suffering from disease or disorders associated with stem cells senescence resulting from a traumatic injury or natural aging.

In a further particular embodiment, subjects according to the invention are subjects suffering from aging-associated tissue dysfunction, for example muscle degeneration and sarcopenia, neural degeneration, indicated as loss of function of sight, hearing, taste, small, and feeling, loss of voluntary and involuntary functions, loss of function to think and reason and/or dysfunction of skin system, indicated as hair loss and greying, reduction of skin thickness, strength, moisture, and elasticity.

In another particular embodiment, subjects according to the invention are stem cells depleted subjects resulting from continuous tissue regeneration due to diseases or aging.

In an embodiment, subjects according to the invention are subjects undergoing graft transplantation.

According to a particular aspect, the agent of the invention is thiamphenicol (TAP) or an analogue thereof.

According to a further aspect of the invention, the agent of the invention is thiamphenicol (TAP).

According to another aspect, the agent of the invention if tetracycline or an analogue thereof.

References cited herein are hereby incorporated by reference in their entirety. The present invention is not to be limited in scope by the specific embodiments and drawings described herein, which are intended as single illustrations of individual aspects of the invention, and functionally equivalent methods and components are within the scope of the invention. The examples illustrating the invention are not intended to limit the scope of the invention in any way.

### EXAMPLES

CASP3 (caspase-3), Cdkn1a (cyclin-dependent kinase inhibitor 1A or p21), CTX (cardiotoxin), eMyHC (embryonic myosin heavy chain), GO (gene ontology), GSEA (gene set enrichment analysis), OCR (oxygen consumption rate), OXPHOS (oxidative phosphorylation), UPR^{mt} (mitochondrial unfolded protein response), NES (normalized enrichment scores), NSC (neural stem cells), TA (tibialis anterior), TCA (tricarboxylic acid cycle).

### Example 1: Identification of Mitochondrial Dysfunction as a Biomarker of MuSCs Senescence

To identify the role of mitochondrial function in stem cell senescence and in particular in muscle SCs senescence, MuSCs from young and aged mice were compared. To identify the principal mechanisms initiating MuSC senescence, publically available MuSC gene expression datasets from young (˜3 months) and aged (˜24 months) mice were compared with the use of gene set enrichment analysis. (GSEA; GEO dataset IDs: GSE47177, GSE47401 and GSE47104) as described below.

Bioinformatic Analysis.

Quadriceps microarray data from young and aged mice MuSCs (Price et al., supra; Bernet et al., supra; Liu et al., supra) were analyzed for transcript expression using the Kyoto encyclopedia of genes and genomes (KEGG), gene ontology (GO) or gene set enrichment analysis (GSEA) analysis. Raw microarray data are also publicly available on Gene Expression Omnibus (GEO) database under the accession numbers GSE47177, GSE47401 and GSE47104. All gene expression heat maps were draw using GENE-E software.

Gene Expression Analyses.

Total RNA was extracted from MuSCs by sorting cells directly into TriPure RNA isolation reagent (Roche) or from cultured C2C12 myoblasts using TriPure reagent according to the product manual. Total RNA was transcribed to cDNA using QuantiTect Reverse Transcription Kit (Qiagen). Expression of selected genes was analyzed using the LightCycler480 system (Roche) and LightCycler® 480 SYBR Green I Master reagent (Roche). The acidic ribosomal protein 36b4 gene (ribosomal protein, large, P0, Rplp0) was used as housekeeping reference. Primer sets for quantitative real-time PCR analyses are shown in Table 1 below.

TABLE 1

| Gene | | |
|---|---|---|
| name | Forward primer | Reverse Primer |
| 36b4 | AGATTCGGGATATGCTGTTGG | AAAGCCTGGAAGAAGGAGGTC |
| | SEQ ID NO: 1 | SEQ ID NO: 2 |
| Ndufb5 | CTTCGAACTTCCTGCTCCTT | GGCCCTGAAAAGAACTACG |
| | SEQ ID NO: 3 | SEQ ID NO: 4 |
| Sdha | GGAACACTCCAAAAACAGAC | CCACCACTGGGTATTGAGTAG |
| | CT | AA |
| | SEQ ID NO: 5 | SEQ ID NO: 6 |
| Sdhc | GCTGCGTTCTTGCTGAGACA | ATCTCCTCCTTAGCTGTGGTT |
| | SEQ ID NO: 7 | SEQ ID NO: 8 |
| Cox5b | AAGTGCATCTGCTTGTCTCG | GTCTTCCTTGGTGCCTGAAG |
| | SEQ ID NO: 9 | SEQ ID NO: 10 |
| Atp5b | GGTTCATCCTGCCAGAGACTA | AATCCCTCATCGAACTGGACG |
| | SEQ ID NO: 11 | SEQ ID NO: 12 |
| Mdh2 | TTGGGCAACCCCTTTCACTC | GCCTTTCACATTTGCTCTGGT |
| | SEQ ID NO: 13 | C |
| | | SEQ ID NO: 14 |
| Idh2 | GGAGAAGCCGGTAGTGGAGAT | GGTCTGGTCACGGTTTGGAA |
| | SEQ ID NO: 15 | SEQ ID NO: 16 |
| Idh3a | CCCATCCCAGTTTGATGTTC | ACCGATTCAAAGATGGCAAC |
| | SEQ ID NO: 17 | SEQ ID NO: 18 |
| Cdkn1a | GTGGGTCTGACTCCAGCCC | CCTTCTCGTGAGACGCTTAC |
| | SEQ ID NO: 19 | SEQ ID NO: 20 |
| Mki67 | TTGGAAAGGAACCATCAAGG | TTTCTGCCAGTGTGCTGTTC |
| | SEQ ID NO: 21 | SEQ ID NO: 22 |
| Cdk4 | CCGGTTGAGACCATTAAGGA | CACGGGTGTTGCGTATGTAG |
| | SEQ ID NO: 23 | SEQ ID NO: 24 |
| Ccna2 | AAGAGAATGTCAACCCCGAAA | ACCCGTCGAGTCTTGAGCTT |
| | SEQ ID NO: 25 | SEQ ID NO: 26 |
| Ccnd1 | GAGCGTGGTGGCTGCGATGC | GGCTTGACTCCAGAAGGGCTT |
| | AA | TCAA |
| | SEQ ID NO: 27 | SEQ ID NO: 28 |
| Ccne1 | CAAAGCCCAAGCAAAGAAAG | CCACTGTCTTTGGAGGCAAT |
| | SEQ ID NO: 29 | SEQ ID NO: 30 |
| Cdc6 | GACACAAGCTACCATGGTTT | CAGGCTGGACGTTTCTAAGTT |
| | SEQ ID NO: 31 | SEQ ID NO: 32 |
| IL6 | GGTGACAACCACGGCCTTCCC | AAGCCTCCGACTTGTGAAGTG |
| | SEQ ID NO: 33 | GT |
| | | SEQ ID NO: 34 |
| IL18 | GTGAACCCCAGACCAGACTG | CCTGGAACACGTTTCTGAAAG |
| | SEQ ID NO: 35 | A |
| | | SEQ ID NO: 36 |
| Hsp60 | ACAGTCCTTCGCCAGATGAGA | TGGATTAGCCCCTTTGCTGA |
| | C | SEQ ID NO: 38 |
| | SEQ ID NO: 37 | |
| Hsp10 | CTGACAGGTTCAATCTCTCCA | AGGTGGCATTATGCTTCCAG |
| | C | SEQ ID NO: 40 |
| | SEQ ID NO: 39 | |
| Clpp | CACACCAAGCAGAGCCTACA | TCCAAGATGCCAAACTCTTG |
| | SEQ ID NO: 41 | SEQ ID NO: 42 |
| Phb | TCGGGAAGGAGTTCACAGAG | CAGCCTTTTCCACCACAAAT |
| | SEQ ID NO: 43 | SEQ ID NO: 44 |
| Phb2 | CAAGGACTTCAGCCTCATCC | GCCACTTGCTTGGCTTCTAC |
| | SEQ ID NO: 45 | SEQ ID NO: 46 |

Animals.

Young (1 month old) and aged (20-24 months old) C57BL/6JRj mice, purchased from Janvier Labs, and five weeks old male C57BL/10SnJ mice or C57BL/10ScSn-Dmdmdx/J, purchased from The Jackson Laboratory, were fed with pellets containing vehicle or NR (400 mg/kg/day) for 6-8 weeks. The pellets were prepared by mixing powdered chow diet (D12450B, Research Diets Inc.) with water or with NR dissolved in water. Pellets were dried under a laminar flow hood for 48 hours. All mice were housed in micro-isolator cages in a room illuminated from 7:00 am-7:00 pm with ad libitum access to diet and water.

FACS Based Skeletal Muscle Stem Cell Isolation.

Gastrocnemius, soleus, quadriceps, and tibialis anterior muscles from both limbs were excised and transferred into PBS on ice. All muscles were trimmed, minced and digested with 0.1 mg/ml of type II collagenase (Sigma) in PBS for 15 min at 37° C. Samples were then centrifuged at 750 g for 5 min and further digested in 1 mg/ml of collagenase/dispase (Roche) for 30 mins at 37° C. Muscle slurries were sequentially filtered through 100, 70 and 40 μm cell strainers. The isolated cells were then washed in washing buffer (PBS+2.5% FBS) then resuspended in 200 μl of washing buffer and immediately stained with antibodies, including the MuSC markers CD31 (1:800, eBioscience, eFluor450 conjugated); CD34 (1:200, eBioscience, eFluor660 conjugated); CD45 (1:200, eBioscience, eFluor450 conjugated); CD11b (1:400, eBioscience, eFluor450 conjugated); Sca-1 (1:1000, eBioscience, PE-Cy7 conjugated); and α7 integrin (1:300, MBL) for 30 min at 4° C. Secondary staining was performed with a mixture of goat anti-mouse antibody (1:800, Life technologies, Alexa Fluor 488 conjugated) and propidium Iodide (PI, Sigma) for 15 min at 4° C. in the dark. Stained cells were analysed and sorted using the FACSAria II instrument (BD Biosciences). Debris and dead cells were excluded by forward scatter, side scatter and PI gating. Cells were sorted either directly on slides for immunostaining or into lysis buffer for NAD+ measurements and into TriPure (Roche) reagent for RNA extraction.

Respirometry on MuSCs.

Basal and uncoupled oxygen consumption rates (OCRs) were measured using the Seahorse extracellular flux bioanalyzer (XF96, Seahorse Bioscience Inc.). To uncouple mitochondria, 5 uM of FCCP was injected after a basal respiration measurement. All measurements were performed in triplicates and results were normalized to total cell number seeded (primary MuSCs) assessed using a Bradford kit (Bio-Rad).

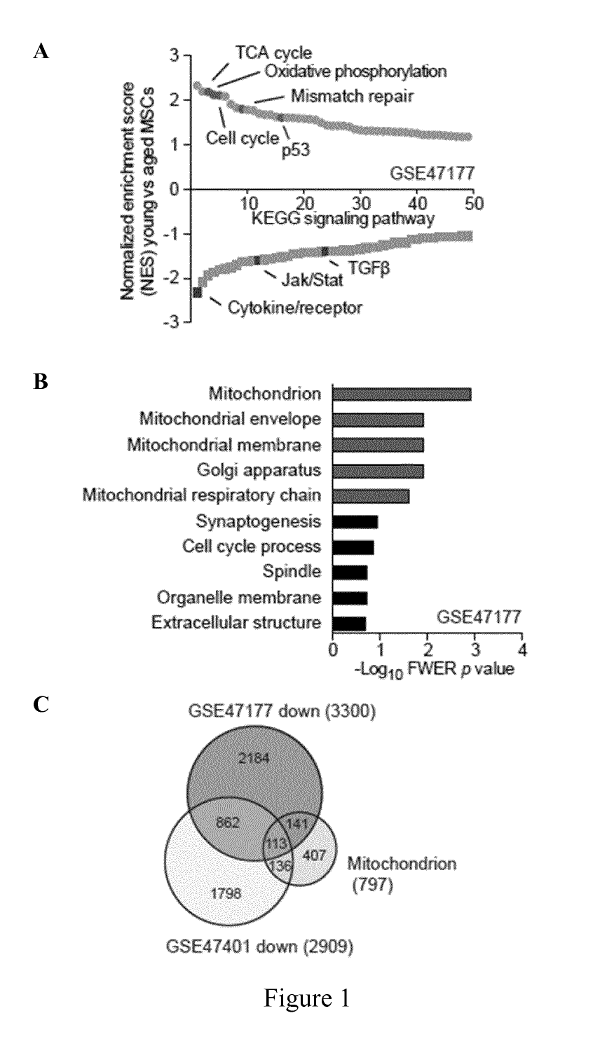

Enrichment scores of young versus aged datasets demonstrate the upregulation of senescence pathways and downregulation of cell cycle pathways with age (FIG. 1A) that is consistent with the paradigm that irreversible cell cycle arrest is a primary marker of cellular senescence (Kuilman et al., supra; Lopez-Otin et al., supra). In all three datasets, citric acid cycle (TCA, also known as the tricarboxylic acid cycle or the Krebs cycle) and oxidative phosphorylation (OXPHOS) pathways were amongst the most downregulated pathways in aged MuSCs, despite the general assumption that MuSCs predominantly rely on glycolysis (FIG. 1A). Gene ontology (GO) term analysis, of genes significantly (p<0.05) downregulated in aged MuSCs, further demonstrated that many of these pathways were related to mitochondrial function (FIG. 1B). Common downregulated genes during aging indicated a substantial overlap (113 genes; 11.59%) with mitochondrial genes (mitochondrial genes as in Mercer et al., 2011, Cell, 146: 645) (FIG. 1C) in contrast to the minimal (11 genes; 1.92%) overlap amongst common upregulated genes. Among the 113 downregulated mitochondrial genes in aged MuSCs, 41.6% were related to the TCA cycle and OXPHOS (FIG. 1D), which is significantly higher than their percent composition of the whole mitochondrial proteome (˜14%) (Sickmann et al., 2003, PNAS, 100: 13207; Pagliarini et al., 2008, Cell, 134: 112). This indicates a dominant decline of mitochondrial respiratory genes in aged MuSCs. The reduction in mitochondrial OXPHOS and TCA cycle genes is consistent for all independent datasets (FIG. 1E).

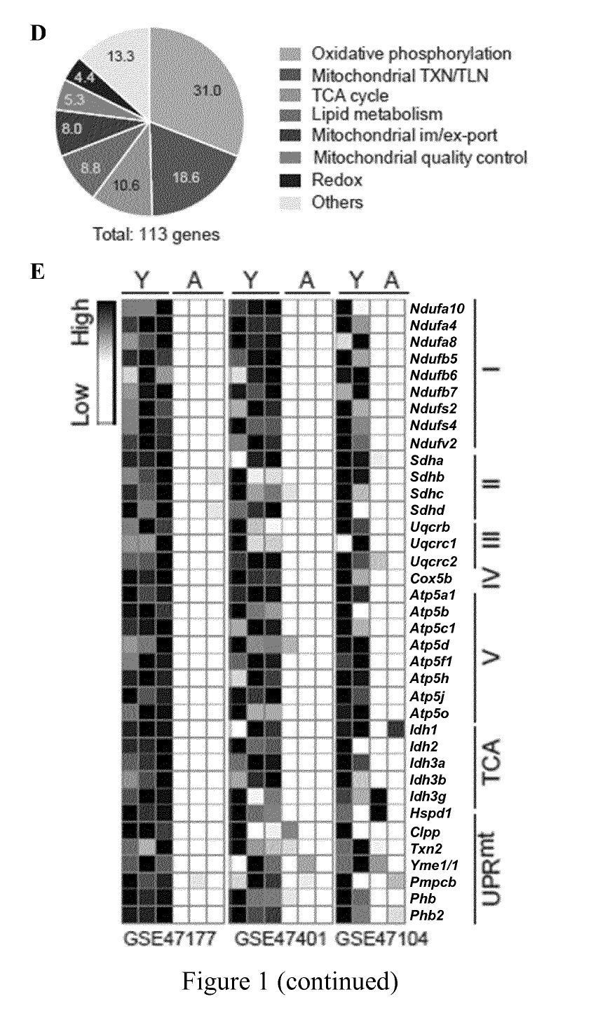

Confirming dysfunctional mitochondrial respiration, isolated primary aged and young MuSCs were isolated. Reductions in OXPHOS and TCA cycle transcripts were found (FIG. 2A), matched by a reduction in oxidative respiration rates (FIG. 2B). Interestingly, several important markers and regulators of the mitochondrial unfolded protein response (UPR^{mt}), a stress response pathway that mediates adaptations in mitochondrial content and function, were significantly downregulated in aged MuSCs (FIGS. 1E and 2C, D). Notably, despite the absence of consistent changes in CDKN2A or MAPK14 (p38) pathways, previously reported to regulate MuSC senescence, there was a downregulation of cell cycle-related gene expression (FIG. 2D). The reduction in cell cycle signalling was accompanied by an upregulation of the cyclin-dependent kinase inhibitor 1A (CDKN1A)-mediated pathway (FIG. 2D), suggesting that early senescence in MuSCs may involve CDKN1A.

These data show that a dominant decline mitochondrial OXPHOS and TCA cycle genes can be observed in aged MuSCs.

### Example 2: Thiamphenicol Induces UPR^{mt} in Myoblasts

The ability of TAP in inhibiting or reversing SC senescence and in particular MuSC senescence is tested in myoblast as follows.

Western Blotting.

C2C12 cells were lysed in a buffer composed of 50 mM Tris, 150 mM KCl, EDTA 1 mM, NP40 1%, nicotinamide 5 mM, sodium butyrate 1 mM and protease inhibitors cocktail (Roche) at pH 7.4. Proteins were separated by SDS-PAGE and transferred onto nitrocellulose membranes. Blocking and antibody incubations were performed in 3% BSA. The following primary antibodies were used: anti-cleavage caspase 3 (Cell Signalling); Anti-γH2AX (Millipore); anti-β-actin (Sigma). All secondary antibodies were from Jackson Immunoresearch. Antibody detection reactions were developed by enhanced chemiluminescence (Advansta, CA, USA) using x-ray films or imaged using the c300 imaging system (Azure Biosystems).

The following primary antibodies were used: anti-HSP60 (Enzo Life Science); anti-PHB (Biolegend); anti-PHB2 (Santa Cruz); anti-CKD4 (Novus biologicals); anti-CCND1 (Santa Cruz); anti-CCND3 (Santa Cruz); anti-HSP90 (BD Biosciences); HSP70 (Abcam); anti-MT-CO1 (Biolegend); anti-ATP5A (Biolegend); anti-Grp78 (Abcam); and anti-CLPP (Sigma).

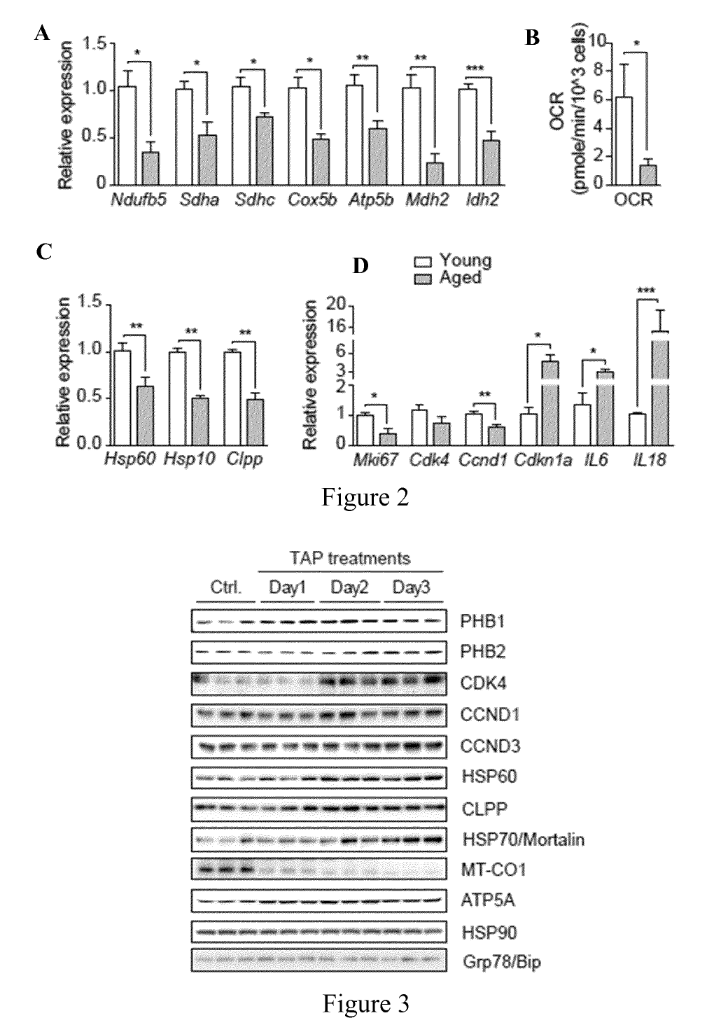

UPR^{mt} induction by thiamphenicol (TAP) which also induced prohibitins and cell cycle gene expression in C2C12 cells (FIG. 3) is supporting the ability of this agent in attenuating the senescence-signaling cascade in those cells.

These results indicate that TAP would be able to reverse SC senescence and in particular MuSC senescence.

Sequence listing

Nucleic acid sequence of 36b4 forward primer
SEQ ID NO: 1: AGATTCGGGATATGCTGTTGG
Nucleic acid sequence of 36b4 reverse primer
SEQ ID NO: 2: AAAGCCTGGAAGAAGGAGGTC
Nucleic acid sequence of Ndufb5 forward primer
SEQ ID NO: 3: CTTCGAACTTCCTGCTCCTT
Nucleic acid sequence of Ndufb5 reverse primer
SEQ ID NO: 4: GGCCCTGAAAAGAACTACG
Nucleic acid sequence of Sdha forward primer
SEQ ID NO: 5: GGAACACTCCAAAAACAGACCT
Nucleic acid sequence of Sdha reverse primer
SEQ ID NO: 6: CCACCACTGGGTATTGAGTAGAA
Nucleic acid sequence of Sdhc forward primer
SEQ ID NO: 7: GCTGCGTTCTTGCTGAGACA
Nucleic acid sequence of Sdhc reverse primer
SEQ ID NO: 8: ATCTCCTCCTTAGCTGTGGTT
Nucleic acid sequence of Cox5b forward primer
SEQ ID NO: 9: AAGTGCATCTGCTTGTCTCG
Nucleic acid sequence of Cox5b reverse primer
SEQ ID NO: 10: GTCTTCCTTGGTGCCTGAAG
Nucleic acid sequence of Atp5b forward primer
SEQ ID NO: 11: GGTTCATCCTGCCAGAGACTA
Nucleic acid sequence of Atp5b reverse primer
SEQ ID NO: 12: AATCCCTCATCGAACTGGACG
Nucleic acid sequence of Mdh2 forward primer
SEQ ID NO: 13: TTGGGCAACCCCTTTCACTC
Nucleic acid sequence of Mdh2 reverse primer
SEQ ID NO: 14: GCCTTTCACATTTGCTCTGGTC
Nucleic acid sequence of Idh2 forward primer
SEQ ID NO: 15: GGAGAAGCCGGTAGTGGAGAT
Nucleic acid sequence of Idh2 reverse primer
SEQ ID NO: 16: GGTCTGGTCACGGTTTGGAA
Nucleic acid sequence of Idh3a forward primer
SEQ ID NO: 17: CCCATCCCAGTTTGATGTTC
Nucleic acid sequence of Idh3a reverse primer
SEQ ID NO: 18: ACCGATTCAAAGATGGCAAC
Nucleic acid sequence of Cdkn1a forward primer
SEQ ID NO: 19: GTGGGTCTGACTCCAGCCC
Nucleic acid sequence of Cdkn1a reverse primer
SEQ ID NO: 20: CCTTCTCGTGAGACGCTTAC
Nucleic acid sequence of Mki67 forward primer
SEQ ID NO: 21: TTGGAAAGGAACCATCAAGG
Nucleic acid sequence of Mki67 reverse primer
SEQ ID NO: 22: TTTCTGCCAGTGTGCTGTTC
Nucleic acid sequence of Cdk4 forward primer
SEQ ID NO: 23: CCGGTTGAGACCATTAAGGA
Nucleic acid sequence of Cdk4 reverse primer
SEQ ID NO: 24: CACGGGTGTTGCGTATGTAG
Nucleic acid sequence of Ccna2 forward primer
SEQ ID NO: 25: AAGAGAATGTCAACCCCGAAA
Nucleic acid sequence of Ccna2 reverse primer
SEQ ID NO: 26: ACCCGTCGAGTCTTGAGCTT
Nucleic acid sequence of Ccnd1 forward primer
SEQ ID NO: 27: GAGCGTGGTGGCTGCGATGCAA
Nucleic acid sequence of Ccnd1 reverse primer
SEQ ID NO: 28: GGCTTGACTCCAGAAGGGCTTCAAT
Nucleic acid sequence of Ccne1 forward primer
SEQ ID NO: 29: CAAAGCCCAAGCAAAGAAAG
Nucleic acid sequence of Ccne1 reverse primer
SEQ ID NO: 30: CCACTGTCTTTGGAGGCAAT
Nucleic acid sequence of Cdc6 forward primer
SEQ ID NO: 31: GACACAAGCTACCATGGTTT
Nucleic acid sequence of Cdc6 reverse primer
SEQ ID NO: 32: CAGGCTGGACGTTTCTAAGTT
Nucleic acid sequence of IL6 forward primer
SEQ ID NO: 33: GGTGACAACCACGGCCTTCCC
Nucleic acid sequence of IL6 reverse primer
SEQ ID NO: 34: AAGCCTCCGACTTGTGAAGTGGT
Nucleic acid sequence of IL18 forward primer
SEQ ID NO: 35: GTGAACCCCAGACCAGACTG
Nucleic acid sequence of IL18 reverse primer
SEQ ID NO: 36: CCTGGAACACGTTTCTGAAAGA
Nucleic acid sequence of Hsp60 forward primer
SEQ ID NO: 37: ACAGTCCTTCGCCAGATGAGAC
Nucleic acid sequence of Hsp60 reverse primer
SEQ ID NO: 38: TGGATTAGCCCCTTTGCTGA
Nucleic acid sequence of Hsp10 forward primer
SEQ ID NO: 39: CTGACAGGTTCAATCTCTCCAC
Nucleic acid sequence of Hsp10 reverse primer
SEQ ID NO: 40: AGGTGGCATTATGCTTCCAG
Nucleic acid sequence of Clpp forward primer
SEQ ID NO: 41: CACACCAAGCAGAGCCTACA
Nucleic acid sequence of Clpp reverse primer
SEQ ID NO: 42: TCCAAGATGCCAAACTCTTG
Nucleic acid sequence of Phb forward primer
SEQ ID NO: 43: TCGGGAAGGAGTTCACAGAG
Nucleic acid sequence of Phb reverse primer
SEQ ID NO: 44: CAGCCTTTTCCACCACAAAT
Nucleic acid sequence of Phb2 forward primer
SEQ ID NO: 45: CAAGGACTTCAGCCTCATCC
Nucleic acid sequence of Phb2 reverse primer
SEQ ID NO: 46: GCCACTTGCTTGGCTTCTAC

1-19. (canceled)

20. A method of preventing and/or treating of diseases or disorders associated with SC senescence and/or decreased SCs number and/or for promoting tissue growth and/or repair, said method comprising administering an effective amount of an agent selected from thiamphenicol (TAP) or an analogue thereof and tetracycline or an analogue thereof.

21. The method according to claim 20, wherein the disease or disorder is characterized by skeletal muscle stem cell senescence.

22. The method according to claim 20, wherein the disease or disorder is characterized by neural stem cell senescence.

23. The method according to claim 20, wherein the disease or disorder is characterized by melanocyte stem cell senescence.

24. The method according to claim 20, wherein said agent is to be administered orally.

25. The method according to claim 20, wherein said agent is to be administered parenterally.

26. The method according to claim 20, wherein said agent is thiamphenicol (TAP) or an analogue thereof.

27. An ex-vivo method for promoting tissue growth and/or increasing engraftment ability of a stem cell containing sample, said method comprising contacting or administering to a stem cell or an isolated tissue or organ, an agent selected from thiamphenicol (TAP) or an analogue thereof and tetracycline or an analogue thereof or a mixture thereof or a composition thereof in an amount to stimulate the survival, proliferation and the maintenance of the stemness of the stem cells within the sample.

28. The method according to claim 27, wherein said SC-containing sample is further combined with a tissue or organ to be grafted before grafting.

29. The method according to claim 27, wherein said agent is thiamphenicol (TAP) or an analogue thereof.

30. A stem cell culture medium comprising at least one agent selected from thiamphenicol (TAP) or an analogue thereof and tetracycline or an analogue thereof or a mixture thereof.

31. A pharmaceutical composition comprising at least one agent selected from thiamphenicol (TAP) or an analogue thereof and tetracycline or an analogue thereof or a mixture thereof and further comprising an agent useful for the prevention and/or treatment of diseases or disorders associated with SC senescence and/or for promoting tissue growth and/or repair.

32. A food supplement comprising at least one agent selected from thiamphenicol (TAP) or an analogue thereof or a mixture thereof.

33. A kit for stem cell culture or for preservation of cells, organs and tissues comprising at least one agent selected from thiamphenicol (TAP) or an analogue thereof and tetracycline or an analogue thereof or a mixture thereof or a formulation thereof and instruction for use.


SEQUENCE LISTING (46 sequences)

SEQ ID NO: 1 - DNA, 21 bp, Artificial Sequence
  36b4 forward primer
agattcgggatatgctgttgg

SEQ ID NO: 2 - DNA, 21 bp, Artificial Sequence
  36b4 reverse primer
aaagcctggaagaaggaggtc

SEQ ID NO: 3 - DNA, 20 bp, Artificial Sequence
  Ndufb5 forward primer
cttcgaacttcctgctcctt

SEQ ID NO: 4 - DNA, 19 bp, Artificial Sequence
  Ndufb5 reverse primer
ggccctgaaaagaactacg

SEQ ID NO: 5 - DNA, 22 bp, Artificial Sequence
  Sdha forward primer
ggaacactccaaaaacagacct

SEQ ID NO: 6 - DNA, 23 bp, Artificial Sequence
  Sdha reverse primer
ccaccactgggtattgagtagaa

SEQ ID NO: 7 - DNA, 20 bp, Artificial Sequence
  Sdhc forward primer
gctgcgttcttgctgagaca

SEQ ID NO: 8 - DNA, 21 bp, Artificial Sequence
  Sdhc reverse primer
atctcctccttagctgtggtt

SEQ ID NO: 9 - DNA, 20 bp, Artificial Sequence
  Cox5b forward primer
aagtgcatctgcttgtctcg

SEQ ID NO: 10 - DNA, 20 bp, Artificial Sequence
  Cox5b reverse primer
gtcttccttggtgcctgaag

SEQ ID NO: 11 - DNA, 21 bp, Artificial Sequence
  Atp5b forward primer
ggttcatcctgccagagacta

SEQ ID NO: 12 - DNA, 21 bp, Artificial Sequence
  Atp5b reverse primer
aatccctcatcgaactggacg

SEQ ID NO: 13 - DNA, 20 bp, Artificial Sequence
  Mdh2 forward primer
ttgggcaacccctttcactc

SEQ ID NO: 14 - DNA, 22 bp, Artificial Sequence
  Mdh2 reverse primer
gcctttcacatttgctctggtc

SEQ ID NO: 15 - DNA, 21 bp, Artificial Sequence
  Idh2 forward primer
ggagaagccggtagtggagat

SEQ ID NO: 16 - DNA, 20 bp, Artificial Sequence
  Idh2 reverse primer
ggtctggtcacggtttggaa

SEQ ID NO: 17 - DNA, 20 bp, Artificial Sequence
  Idh3a forward primer
cccatcccagtttgatgttc

SEQ ID NO: 18 - DNA, 20 bp, Artificial Sequence
  Idh3a reverse primer
accgattcaaagatggcaac

SEQ ID NO: 19 - DNA, 19 bp, Artificial Sequence
  Cdkn1a forward primer
gtgggtctgactccagccc

SEQ ID NO: 20 - DNA, 20 bp, Artificial Sequence
  Cdkn1a reverse primer
ccttctcgtgagacgcttac

SEQ ID NO: 21 - DNA, 20 bp, Artificial Sequence
  Mki67 forward primer
ttggaaaggaaccatcaagg

SEQ ID NO: 22 - DNA, 20 bp, Artificial Sequence
  Mki67 reverse primer
tttctgccagtgtgctgttc

SEQ ID NO: 23 - DNA, 20 bp, Artificial Sequence
  Cdk4 forward primer
ccggttgagaccattaagga

SEQ ID NO: 24 - DNA, 20 bp, Artificial Sequence
  Cdk4 reverse primer
cacgggtgttgcgtatgtag

SEQ ID NO: 25 - DNA, 21 bp, Artificial Sequence
  Ccna2 forward primer
aagagaatgtcaaccccgaaa

SEQ ID NO: 26 - DNA, 20 bp, Artificial Sequence
  Ccna2 reverse primer
acccgtcgagtcttgagctt

SEQ ID NO: 27 - DNA, 22 bp, Artificial Sequence
  Ccnd1 forward primer
gagcgtggtggctgcgatgcaa

SEQ ID NO: 28 - DNA, 25 bp, Artificial Sequence
  Ccnd1 reverse primer
ggcttgactccagaagggcttcaat

SEQ ID NO: 29 - DNA, 20 bp, Artificial Sequence
  Ccne1 forward primer
caaagcccaagcaaagaaag

SEQ ID NO: 30 - DNA, 20 bp, Artificial Sequence
  Ccne1 reverse primer
ccactgtctttggaggcaat

SEQ ID NO: 31 - DNA, 20 bp, Artificial Sequence
  Cdc6 forward primer
gacacaagctaccatggttt

SEQ ID NO: 32 - DNA, 21 bp, Artificial Sequence
  Cdc6 reverse primer
caggctggacgtttctaagtt

SEQ ID NO: 33 - DNA, 21 bp, Artificial Sequence
  IL6 forward primer
ggtgacaaccacggccttccc

SEQ ID NO: 34 - DNA, 23 bp, Artificial Sequence
  IL6 reverse primer
aagcctccgacttgtgaagtggt

SEQ ID NO: 35 - DNA, 20 bp, Artificial Sequence
  IL18 forward primer
gtgaaccccagaccagactg

SEQ ID NO: 36 - DNA, 22 bp, Artificial Sequence
  IL18 reverse primer
cctggaacacgtttctgaaaga

SEQ ID NO: 37 - DNA, 22 bp, Artificial Sequence
  Hsp60 forward primer
acagtccttcgccagatgagac

SEQ ID NO: 38 - DNA, 20 bp, Artificial Sequence
  Hsp60 reverse primer
tggattagcccctttgctga

SEQ ID NO: 39 - DNA, 22 bp, Artificial Sequence
  Hsp10 forward primer
ctgacaggttcaatctctccac

SEQ ID NO: 40 - DNA, 20 bp, Artificial Sequence
  Hsp10 reverse primer
aggtggcattatgcttccag

SEQ ID NO: 41 - DNA, 20 bp, Artificial Sequence
  Clpp forward primer
cacaccaagcagagcctaca

SEQ ID NO: 42 - DNA, 20 bp, Artificial Sequence
  Clpp reverse primer
tccaagatgccaaactcttg

SEQ ID NO: 43 - DNA, 20 bp, Artificial Sequence
  Phb forward primer
tcgggaaggagttcacagag

SEQ ID NO: 44 - DNA, 20 bp, Artificial Sequence
  Phb reverse primer
cagccttttccaccacaaat

SEQ ID NO: 45 - DNA, 20 bp, Artificial Sequence
  Phb2 forward primer
caaggacttcagcctcatcc

SEQ ID NO: 46 - DNA, 20 bp, Artificial Sequence
  Phb2 reverse primer
gccacttgcttggcttctac